(19) United States
(12) Patent Application Publication
(10) Pub. No.: US 2005/0143463 A1
(43) Pub. Date: Jun. 30, 2005

(54) Method of inhibiting angiogenesis

(75) Inventor: Ravi Krishnan, Royston Park (AU)

Correspondence Address: BIRCH STEWART KOLASCH & BIRCH, PO BOX 747, FALLS CHURCH, VA, 22040-0747, US

(73) Assignee: THE QUEEN ELIZABETH HOSPITAL RESEARCH FOUNDATION INC., South Australia (AU)

(21) Appl. No.: 10/979,177

(22) Filed: Nov. 3, 2004

Related U.S. Application Data
(63) Continuation of application No. PCT/AU03/00522, filed on May 2, 2003.

(30) Foreign Application Priority Data
May 3, 2002 (AU) PS2123
Mar. 3, 2003 (AU) 2003900902

(51) Int. Cl. A61K 31/20

(52) U.S. Cl. 514/558

(57) ABSTRACT

The present invention relates to a method of inhibiting endothelial cell proliferation in a biological system, the method including the step of administering to the biological system an effective amount of an alkyl-substituted fatty acid, wherein the alkyl-substituted fatty acid is capable of inhibiting endothelial cell proliferation.

### FIELD OF THE INVENTION

The present invention relates to methods and compositions for inhibiting angiogenesis.

### BACKGROUND OF THE INVENTION

Angiogenesis is the process in which new blood vessels grow into an area which lacks a sufficient blood supply. The growth of endothelial cells is a critical step in the angiogenic process. Angiogenesis commences with the erosion of the basement membrane surrounding endothelial cells which line the lumen of blood vessels. Erosion of the basement membrane is triggered by enzymes released by endothelial cells and leukocytes. The endothelial cells then migrate through the eroded basement membrane when induced by angiogenic stimulants. The migrating cells form a “sprout” off the parent blood vessel. The migrating endothelial cells proliferate, and the sprouts merge to form capillary loops, thus forming a new blood vessel.

The control of angiogenesis is a highly regulated process involving the actions of a number of angiogenic stimulators and inhibitors. Both controlled and uncontrolled angiogenesis are thought to proceed in a similar manner.

Under normal physiological conditions, humans and animals only undergo angiogenesis in very specific restricted situations. For example, angiogenesis is only normally observed in wound healing, foetal and embryonic development, and formation of the corpus luteum, endometrium and placenta.

However, uncontrolled or undesired angiogenesis is associated with many diseases and conditions. For example, angiogenesis plays a pivotal role in tumour formation and expansion, and also in the cornea and retina of patients with certain ocular disorders.

The evidence for the role of angiogenesis in tumour growth is extensive. It is generally accepted that the growth of tumours is critically dependent upon this process. Angiogenesis plays a critical role in two stages of tumour development. Firstly, angiogenesis is required for a tumour mass to grow beyond a size of a few millimetres. Without the formation of new vasculature, the cells in the tumour mass will not receive sufficient blood supply to develop beyond this small size. However, once vascularization of the tumour commences, the tumour mass may then expand.

Vascularization of the tumour also plays a significant role in the development of secondary tumours. Vascularization of the tumour allows tumour cells to enter the blood stream and to circulate throughout the body. After the tumour cells have left the primary site and settled into a secondary (metastatic) site, further angiogenesis then allows the secondary tumour mass to grow and expand. Therefore, prevention of angiogenesis may not only lead to a reduction in the growth of a tumour at its primary site, but the prevention of angiogenesis may also reduce the loss of cells from the primary site that may go on to form metastases.

In addition to the formation tumors, there are also various diseases and conditions induced by angiogenesis or associated with uncontrolled or undesired angiogenesis, including diabetic retinopathy, retrolental fibroplasia, neovascular glaucoma, psoriasis, angiofibroma, immune and nonimmune inflammation (including rheumatic arthritis), the propagation of capillary vessels in arteriosclerosis plaques, angioma and Kaposi's sarcoma. Angiogenesis can also occur in a rheumatoid joint, hastening joint destruction by allowing an influx of leukocytes with subsequent release of inflammatory mediators.

One example of a disease mediated by angiogenesis is ocular neovascular disease. This disease is characterized by invasion of new blood vessels into the structures of the eye such as the retina or cornea. It is the most common cause of blindness and is associated with a large number of diseases of the eye. In age-related macular degeneration, the associated visual problems are caused by an ingrowth of choroidal capillaries through defects in Bruch's membrane with proliferation of fibrovascular tissue beneath the retinal pigment epithelium.

Chronic inflammation may also involve pathological angiogenesis. Such disease states as ulcerative colitis and Crohn's disease show histological changes with the ingrowth of new blood vessels into the inflamed tissues. Another pathological role associated with angiogenesis is found in atherosclerosis. The plaques formed within the lumen of blood vessels have been shown to have angiogenic stimulatory activity.

Angiogenesis is also involved in reproduction and wound healing. In reproduction, angiogenesis is an important step in ovulation and also in implantation of the blastula after fertilization. Prevention of angiogenesis may be used to induce amenorrhea, to block ovulation, or to prevent implantation by the blastula. In wound healing, excessive repair or fibroplasia can be a detrimental side effect of surgical procedures and may be caused or exacerbated by angiogenesis. Adhesions are a frequent complication of surgery and lead to problems such as small bowel obstruction.

The current treatment of diseases involving uncontrolled or undesired angiogenesis is inadequate. Accordingly, there is a need for new methods and compositions that inhibit uncontrolled or undesired angiogenesis.

The present invention relates to the identification of a class of agents that act to inhibit angiogenesis. In particular, the present invention relates to methods of inhibiting angiogenesis, and pharmaceutical compositions suitable for inhibiting angiogenesis.

### SUMMARY OF THE INVENTION

The present invention provides a method of inhibiting endothelial cell proliferation in a biological system, the method including the step of administering to the biological system an effective amount of an alkyl-substituted fatty acid, wherein the alkyl-substituted fatty acid is capable of inhibiting endothelial cell proliferation and the alkyl-substituted fatty acid has the following chemical formula: 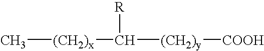
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

The present invention also provides a method of inhibiting angiogenesis in a biological system, the method including the step of administering to the biological system an effective amount of an alkyl-substituted fatty acid, wherein the alkyl-substituted fatty acid is capable of inhibiting angiogenesis and the alkyl-substituted fatty acid has the following chemical formula: 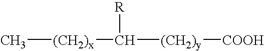
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

The present invention also provides a method of reducing the amount of an agent administered to a biological system to achieve a desired level of inhibition of endothelial cell proliferation, the method including the step of administering to the biological system an effective amount of an alkyl-substituted fatty acid, wherein the alkyl-substituted fatty acid has the following chemical formula: 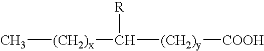
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH=CH group or a C≡C group, and x+y is between 2 and 46.

The present invention also provides a method of reducing the amount of an anti-angiogenic agent administered to a biological system to achieve a desired level of inhibition of angiogenesis, the method including the step of administering to the biological system an effective amount of an alkyl-substituted fatty acid, wherein the alkyl-substituted fatty acid has the following chemical formula: 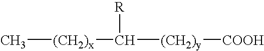
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

The present invention further provides a pharmaceutical composition including an alkyl-substituted fatty acid, wherein the alkyl-substituted fatty acid is capable of inhibiting endothelial cell proliferation and/or angiogenesis and the alkyl-substituted fatty acid has the following chemical formula: 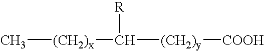
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH=CH group or a C≡C group, and x+y is between 2 and 46.

The present invention also provides a pharmaceutical composition including an alkyl-substituted fatty acid and an immunosuppressant, wherein the alkyl-substituted fatty acid has the following chemical formula: 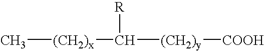
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

The present invention arises out of studies into the ability of alkyl-substituted fatty acids to inhibit the proliferation of human umbilical vein endothelial cells (HUVECs). In particular, it has been surprisingly found that the alkyl-substituted fatty acids 16-methyl heptadecanoic acid, 15-methyl heptadecanoic acid, 15-methyl hexadecanoic acid, 14-methyl hexadecanoic acid, 14-methyl pentadecanoic acid, 13-methyl pentadecanoic acid, 13-methyl tetradecanoic acid, 12-methyl tetradecanoic acid, 12-methyl tridecanoic acid, 11-methyl tridecanoic acid, 11-methyl dodecanoic acid, and 10-methyl undecanoic acid have the capacity to inhibit the proliferation of human umbilical vein endothelial cells (HUVECs) in vitro in a dose dependent manner. In addition, the alkyl-substituted fatty acids 12-methyltetradecanoic acid, 13-methyltetradecanoic acid, 14-methylpentadecanoic acid, 10-methyloctadecanoic acid, 17-methyloctadecanoic acid and 16-methyltetradecanoic acid inhibit angiogenesis in a chicken chorioallantoic membrane (CAM) assay in a dose dependent manner. The toxicity of these alkyl-substituted fatty acids in this angiogenesis assay is low, demonstrating that these alkyl-substituted fatty acids have significant therapeutic potential. Finally, the alkyl-substituted fatty acid 12-methlytetradecanoic acid inhibits corneal neovascularisation in mice.

Various terms that will be used throughout the specification have meanings that will be well understood by a skilled addressee. However, for ease of reference, some of these terms will now be defined.

The term “alkyl-substituted fatty acid” as used throughout the specification is to be understood to mean any branched fatty acid that may be described by the following chemical formula:

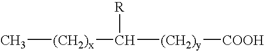

Where R is an alkyl group of 1 to 6 carbon atoms. For alkyl-substituted saturated fatty acids, x is equal to or greater than 0, y is equal to or greater than 0, and x+y is between 0 and 46. For alkyl-substituted unsaturated fatty acids, x or y is equal to or greater than 2, at least one CH₂—CH₂ group in (CH₂)ₓ and/or (CH₂)_{y} is replaced with a CH═CH group or a C≡C group, and x+y is between 2 and 46.

As will be appreciated, the term “alkyl-substituted fatty acid” includes within its scope any salts of the carboxylic acid, or any derivatives of the compounds according to the above chemical formula that are functionally equivalent to the compounds in terms of their ability to inhibit endothelial cell proliferation and/or inhibit angiogenesis.

The term “angiogenesis” as used throughout the specification is to be understood to mean the generation of new blood vessels (“neovascularization”), for example into a tissue or organ.

The term “inhibit” as used throughout the specification is to be understood to mean a reduction in the progress of a process, including the start, continuation or termination of a process. Such processes include, for example, the proliferation of endothelial cells or the angiogenic process itself.

The term “biological system” as used throughout the specification is to be understood to mean any multi-cellular system and includes isolated groups of cells to whole organisms. For example, the biological system may be cells in tissue culture, a tissue or organ, or an entire human subject suffering the effects of undesired or uncontrolled angiogenesis, or a disease or condition associated with uncontrolled or undesired angiogenesis.

The term “anti-angiogenic agent” as used throughout the specification is to be understood to mean any agent that has the capacity to inhibit angiogenesis in a biological system.

The term “immunosuppressant” as used throughout the specification is to be understood to mean any agent that can modify the immune response and/or surveillance, such that the response of immune cells towards alloantigens, autoantigens, xenoantigens or inflammatory mediators is reduced.

The term “immunophilin” as used throughout the specification is to be understood to mean receptors that bind to the class of immunosuppressants that includes cyclosporin A, rapamycin and FK506.

### BRIEF DESCRIPTION OF THE FIGURES

FIG. 1 shows the extent of angiogenesis in chorioallantoic membranes treated with varying doses of 12-MTA.

FIG. 2 shows in the top panel the extent of angiogenesis in chorioallantoic membranes treated with 25 nmol or 100 nmol of 17-MODA. The lower panel shows the extent of angiogenesis in chorioallantoic membranes treated with 100 nmol of 10-MODA.

FIG. 3 shows in the top panel the extent of angiogenesis in chorioallantoic membranes treated with 100 nmol of 14-MPDA. The lower panel shows the extent of angiogenesis in chorioallantoic membranes treated with 100 nmol of 13-MTA.

FIG. 4 shows the extent of angiogenesis in chorioallantoic membranes treated with 100 nmol of 16-MTA.

FIG. 5 shows the extent of corneal neovascularisation in the cornea of mice that were scratched and treated with pseudomonas aeruginosa to induce corneal neovascularisation after treatment with 12-MTA for 7 days and 14 days.

FIG. 6 shows histological examination of corneas treated with vehicle or 12-MTA 14 days post challenge with pseudomonas aeruginosa to induce corneal neovascularisation.

### GENERAL DESCRIPTION OF THE INVENTION

As mentioned above, in one form the present invention provides a method of inhibiting endothelial cell proliferation in a biological system, the method including the step of administering to the biological system an effective amount of an alkyl-substituted fatty acid, wherein the alkyl-substituted fatty acid is capable of inhibiting endothelial cell proliferation and the allyl-substituted fatty acid has the following chemical formula: 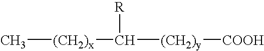
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH=CH group or a C≡C group, and x+y is between 2 and 46.

The endothelial cell is any endothelial cell that is undergoing proliferation, including an endothelial cell undergoing proliferation in response to one or more angiogenic stimuli in a biological system, or endothelial cells that have the capacity to undergo proliferation in response to one or more angiogenic stimuli. Preferably, the endothelial cell proliferation is associated with angiogenesis in the biological system. More preferably, the endothelial cell proliferation is associated with uncontrolled or undersired angiogenesis in the biological system.

Preferably, the endothelial cell is a human or animal endothelial cell. Most preferably, the endothelial cell is a human endothelial cell.

Preferably, the endothelial cell is undergoing proliferation associated with a disease or condition in a human or an animal that is associated with uncontrolled or undesired angiogenesis. More preferably, the endothelial cell is undergoing proliferation associated with one or more of the following diseases or conditions in a human or animal: angiogenesis associated with solid tumours; angiofibroma; corneal neovascularisation; retinal/choroidal neovascularization; arteriovenous malformations; arthritis, including rheumatoid arthritis, lupus and other connective tissue disorders; Osler-Weber syndrome; atherosclerotic plaques; psoriasis; pyogenic granuloma; retrolental fibroplasias; scleroderma; granulations, hemangioma; trachoma; hemophilic joints; vascular adhesions and hypertrophic scars; diseases associated with chronic inflammation including sarcoidosis and inflammatory bowel diseases such as Crohn's disease and ulcerative colitis. More preferably, the angiogenesis is associated with corneal neovascularisation, retinal neovascularisation or choroidal neovascularisation. Most preferably, the angiogenesis is associated with corneal neovascularisation.

Diseases associated with corneal neovascularization include diabetic retinopathy, retinopathy of prematurity, corneal graft rejection, neovascular glaucoma and retrolental fibroplasia, epidemic keratoconjunctivitis, Vitamin A deficiency, contact lens overwear, atopic keratitis, superior limbic keratitis, pterygium keratitis sicca, Sjogrens Syndrome, acne rosacea, phylectenulosis, syphilis, mycobacteria infections, lipid degeneration, chemical burns, bacterial ulcers, fungal ulcers, herpes simplex infections, herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, scleritis, Stevens-Johnson disease, pemphigoid, and radial keratotomy.

Diseases associated with retinal/choroidal neovascularization include diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/nitritis, mycobacterial infections, Lyme's disease, systemic lupus erythematosis, retinopathy of prematurity, Eales' disease, Behcet's disease, infections causing a retinibs or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis and diseases caused by the abnormal proliferation of fibrovascular or fibrous tissue including all forms of proliferative vitreoretinopathy.

The biological system is any system that includes endothelial cells that have the capacity to proliferate, or any system that includes endothelial cells that are proliferating. Preferably, the biological system is a human or animal subject that includes endothelial cells that have the capacity to proliferate, or endothelial cells that are proliferating. More preferably, the biological system is a human or animal subject that includes the proliferation of endothelial cells associated with a disease or condition that is due to undesired or uncontrolled angiogenesis. More preferably, the biological system is a human or animal subject suffering from a disease or condition involving the proliferation of endothelial cells. Most preferably, the biological system is a human or animal subject suffering from one or more of the following diseases or conditions associated with the proliferation of endothelial cells: angiogenesis associated with solid tumours; angiofibroma; corneal neovascularisation; retinal/choroidal neovascularization; arteriovenous malformations; arthritis, including rheumatoid arthritis, lupus and other connective tissue disorders; Osler-Weber syndrome; atherosclerotic plaques; psoriasis; pyogenic granuloma; retrolental fibroplasias; scleroderma; granulations, henagioma; trachoma; hemophilic joints; vascular adhesions and hypertrophic scars; diseases associated with chronic inflammation including sarcoidosis and inflammatory bowel diseases such as Crohn's disease and ulcerative colitis.

The alkyl-substituted fatty acid according to the various forms of the present invention is any alkyl-substituted fatty acid described by the following chemical formula:

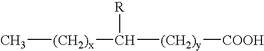

Where R is an alkyl group of 1 to 6 carbon atoms. For alkyl-substituted saturated fatty acids, x is equal to or greater than 0, y is equal to or greater than 0, and x+y is between 0 and 46. For alkyl-substituted unsaturated fatty acids, x or y is equal to or greater than 2, at least one CH₂—CH₂ group in (CH₂)ₓ and/or (CH₂)_{y} is replaced with a CH═CH group or a C≡C group, and x+y is between 2 and 46.

Preferably, the alkyl group (R) in the alkyl-substituted fatty is a methyl or ethyl group. Most preferably, the alkyl group (R) is a methyl group.

Preferably, the alkyl group (R ) in the alkyl-substituted fatty acid is located on the first carbon atom directly adjacent to the terminal methyl group, or on the second carbon removed from the terminal methyl group.

Preferably, the alkyl-substituted fatty acid is a saturated alkyl-substituted fatty acid. More preferably, the saturated alkyl-substituted fatty acid is a derivative of undecanoic acid, dodecanoic acid, tridecanoic acid, tetradecanoic acid, pentadecanoic acid, hexadecanoic acid, heptadecanoic acid, octadecanoic acid, nonadecanoic acid, or eicosanoic acid. Most preferably, the saturated alkyl-substituted fatty acid is a derivative of tetradecanoic acid.

Preferably, the saturated alkyl-substituted fatty acid is 18-methylnonadecanoic acid, 17-methyloctadecanoic acid, 10-methyloctadecanoic acid, 16-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyldodecanoic acid, or any combination of these alkyl-substituted fatty acids.

More preferably, the alkyl-substituted fatty acid is 16-methyl heptadecanoic acid, 15-methyl heptadecanoic acid, 15-methyl hexadecanoic acid, 14-methyl hexadecanoic acid, 14-methyl pentadecanoic acid, 13-methyl pentadecanoic acid, 13-methyl tetradecanoic acid, 12-methyl tetradecanoic acid, 12-methyl tridecanoic acid, 11-methyl tridecanoic acid, 11-methyl dodecanoic acid, 10-methyl undecanoic acid, or any combination of these fatty acids. Most preferably, the alkyl-substituted fatty acid is 12-methyltetradecanoic acid.

Accordingly, in a preferred form, the present invention provides a method of inhibiting endothelial cell proliferation in a biological system, the method including the step of administering to the biological system an effective amount of 16-methyl heptadecanoic acid, 15-methyl heptadecanoic acid, 15-methyl hexadecanoic acid, 14-methyl hexadecanoic acid, 14-methyl pentadecanoic acid, 13-methyl pentadecanoic acid, 13-methyl tetradecanoic acid, 12-methyl tetradecanoic acid, 12-methyl tridecanoic acid, 11-methyl tridecanoic acid, 11-methyl dodecanoic acid, 10-methyl undecanoic acid, or any combination of these alkyl-substituted fatty acids.

With regard to unsaturated alkyl-substituted fatty acids, the unsaturated alkyl-substituted saturated fatty acid is preferably a derivative of undecenoic acid, dodecenoic acid, tridecenoic acid, tetradecenoic acid, pentadecenoic acid, hexadecenoic acid, heptadecenoic acid, octadecenoic acid, nonadecenoic acid, or eicosenoic acid.

The effective amount of alkyl-substituted fatty acid to be administered is not particularly limited, so long as it is within such an amount and in such a form that generally exhibits a pharmacologically useful or therapeutic effect.

In this regard, an effective amount of the alkyl-substituted fatty acid may be appropriately chosen, depending upon the extent of endothelial cell proliferation to be inhibited, the age and body weight of the subject, the frequency of administration, and the presence of other active agents.

Preferably, the effective amount of alkyl-substituted fatty acid administered results in a concentration of the compound at the desired site of action in the biological system in the range from 50 nM to 5 mM. More preferably, the effective amount of alkyl-substituted fatty acid administered results in a concentration of the compound at the desired site of action in the biological system in the range from 50 nM to 1 mM. Most preferably, the effective amount of alkyl-substituted fatty acid administered results in a concentration of the compound at the desired site of action in the biological system in the range from 25 μM to 500 μM.

In the case of topical administration of the alkyl-substituted fatty acid, the effective amount of the alkyl-substituted fatty acid applied topically to a desired site is preferably in the range from 25 nmol to 200 μmol.

The administration of alkyl-substituted fatty acid may be within any time suitable to produce the desired effect of inhibiting the proliferation of endothelial cells. In a human or animal subject, the alkyl-subsututed fatty acid may be administered orally, parenterally, topically or by any other suitable means, and therefore transit time of the drug must be taken into account.

The administration of the alkyl-substituted fatty acid in the various forms of the present invention may also include the use of one or more pharmaceutically acceptable additives, including pharmaceutically acceptable salts, amino acids, polypeptides, polymers, solvents, buffers, excipients and bulking agents, taking into consideration the particular physical and chemical characteristics of the alkyl-substituted fatty acid to be administered.

For example, the alkyl-substituted fatty acid can be prepared into a variety of pharmaceutical preparations in the form of, e.g., an aqueous solution, an oily preparation, a fatty emulsion, an emulsion, a gel, etc., and these preparations can be administered as intramuscular or subcutaneous injection or as injection to the organ, or as an embedded preparation or as a transmucosal preparation through nasal cavity, rectum, uterus, vagina, lung, etc. The composition may be administered in the form of oral preparations (for example solid preparations such as tablets, capsules, granules or powders; liquid preparations such as syrup, emulsions or suspensions). Compositions containing the alkyl-substituted fatty acid may also contain a preservative, stabiliser, dispersing agent, pH controller or isotonic agent. Examples of suitable preservatives are glycerin, propylene glycol, phenol or benzyl alcohol. Examples of suitable stabilisers are dextran, gelatin, α-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

The administration of the alkyl-substituted fatty acid in the various forms of the present invention may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of the particular alkyl-substituted fatty acid.

For these purposes, the composition may be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the composition will normally be in a unit dosage, sterile injectable form (solution, suspension or emulsion) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,3-butanediol. Among the acceptable vehicles and solvents that may be employed are water, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

When administered orally, the composition will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Molded tablets may be made by molding in a suitable machine, a mixture of the powdered active ingredient and a suitable carrier moistened with an inert liquid diluent.

The administration of the alkyl-substituted fatty acid in the various forms of the present invention may also utilize controlled release technology. The alkyl-substituted fatty acid may also be administered as a sustained-release pharmaceutical. To further increase the sustained release effect, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, the alkyl-substituted fatty acid may be incorporated into a hydrophobic polymer matrix for controlled release over a period of days. The composition of the invention may then be molded into a solid implant, or externally applied patch, suitable for providing efficacious concentrations of the alkyl-substituted fatty acid over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time release characteristics and release kinetics. The composition may then be molded into a solid implant suitable for providing efficacious concentrations of the alkyl-substituted fatty acid over a prolonged period of time without the need for frequent re-dosing. The alkyl-substituted fatty acid can be incorporated into the biodegradable polymer or polymer mixture in any suitable manner known to one of ordinary skill in the art and may form a homogeneous matrix with the biodegradable polymer, or may be encapsulated in some way within the polymer, or may be molded into a solid implant.

It has also been surprisingly found that the ability of alkyl-substituted fatty acids to inhibit proliferation of human vein endothelial cells is markedly improved in the presence of immunosuppressants. For example, the ability of the alkyl-substituted fatty acid 12-methyltetradecanoic acid to inhibit proliferation of human vein endothelial cells is further markedly improved in the presence of the immunophilin binding immunosupressants cyclosporin A and rapamycin.

Accordingly, the administration of the alkyl-substituted fatty acid in the various forms of the present invention may further include the administration of an immunosuppressant. Preferably, the immunosuppressant is an agent that binds to an immunophilin. More preferably, the immunosuppressant is cyclosporin A, rapamycin or FK506. Most preferably, the immunosuppressant is rapamycin.

In a preferred form, the present invention provides a method of inhibiting endothelial cell proliferation in a biological system, the method including the step of administering to the biological system an effective amount of rapamycin and 16-methyl heptadecanoic acid, 15-methyl heptadecanoic acid, 15-methyl hexadecanoic acid, 14-methyl hexadecanoic acid, 14-methyl pentadecanoic acid, 13-methyl pentadecanoic acid, 13-methyl tetradecanoic acid, 12-methyl tetradecanoic acid, 12-methyl tridecanoic acid, 11-methyl tridecanoic acid, 11-methyl dodecanoic acid, 10-methyl undecanoic acid, or any combination of these alkyl-substituted fatty acids.

In another preferred form, the present invention provides a method of inhibiting endothelial cell proliferation in a biological system, the method including the step of administering to the biological system an effective amount of cyclosporin A and 16-methyl heptadecanoic acid, 15-methyl heptadecanoic acid, 15-methyl hexadecanoic acid, 14-methyl hexadecanoic acid, 14-methyl pentadecanoic acid, 13-methyl pentadecanoic acid, 13-methyl tetradecanoic acid, 12-methyl tetradecanoic acid, 12-methyl tridecanoic acid, 11-methyl tridecanoic acid, 11-methyl dodecanoic acid, 10-methyl undecanoic acid, or any combination of these alkyl-substituted fatty acids.

An effective amount of the immunosuppressant may be appropriately chosen, depending upon the amount of alkyl-substituted fatty acid in the composition, the extent of endothelial proliferation to be inhibited, the age and body weight of the subject, and the frequency of administration.

In the case of administration of cyclosporin A, preferably this agent is administered so that the concentration of the compound at the desired site of action in the biological system is in the range from 10 nM to 2 μM. More preferably, cyclosporin A is administered so that the concentration of the compound at the desired site of action in the biological system is in the range from 10 nM to 100 nM.

In the case of administration of rapamycin, preferably this agent is administered so that the concentration of the compound at the desired site of action in the biological system is in the range from 0.1 nM to 30 nM. More preferably, rapamycin is administered so that the concentration of the compound at the desired site of action in the biological system is in the range from 0.1 nM to 10 nM.

The administration of immunosuppressant may be within any time suitable to produce the desired effect of inhibiting the proliferation of endothelial cells in conjunction with the alkyl-substituted fatty acid. In a human or animal subject, the immunosuppressant may be administered orally, parenterally, topically or by any other suitable means and therefore transit time of the drug must be taken into account. The administration of the immunosuppressant may occur at the same time and in the same manner as the administration of the alkyl-substituted fatty acid. Alternatively, the administration of the immunosuppressant may be separate to the administration of the alkyl-substituted fatty acid, and occur at a pharmacologically appropriate time before or after administration of the alkyl-substituted fatty acid.

The administration of the immunosuppressant in the various forms of the present invention may also include the use of one or more pharmaceutically acceptable additives, including pharmaceutically acceptable salts, amino acids, polypeptides, polymers, solvents, buffers, excipients and bulking agents.

The inhibition of the proliferation of endothelial cells in the biological system may be determined by a suitable method known in the art, such as cell counting, 3[H] thymidine incorporation, immuno-histochemical staining for cell proliferation, delayed appearance of neovascular structures, slowed development of neovascular structures, decreased occurrence of neovascular structures, slowed or decreased severity of angiogenesis-dependent disease effects, arrested angiogenic growth, or regression of previous angiogenic growth.

The determination of the ability of an alkyl-substituted fatty acid to inhibit proliferation of endothelial cells may be by a suitable assay known in the art in which cells are treated with the alkyl-substituted fatty acid and endothelial cell proliferation measured. For example, human umbilical vascular endothelial cells may be cultured in vitro in the appropriate medium and endothelial cell proliferation may be measured, for example, by trtiated thymidine uptake. The ability of the alkyl-substituted fatty acid (ie the test fatty acid) to inhibit proliferation in such an assay may then be tested by contacting the endothelial cells with the test fatty acid and determining the extent of inhibition of proliferation that occurs at any particular concentration of the test fatty acid.

As will be appreciated, in determining the ability of a test fatty acid to inhibit the proliferation of endothelial cells, the test fatty acid will be delivered at a concentration and in form that are suitable to the particular physical and chemical characteristics of the test fatty acid.

The present invention also provides a method of inhibiting angiogenesis in a biological system, the method including the step of administering to the biological system an effective amount of an alkyl-substituted fatty acid, wherein the alkyl-substituted fatty acid is capable of inhibiting angiogenesis and the alkyl-substituted fatty acid has the following chemical formula: 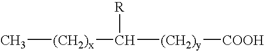
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH=CH group or a C≡C group, and x+y is between 2 and 46.

The angiogenesis may be any angiogenesis occurring in a biological system. Preferably, the angiogenesis occurs in an animal or human subject. Most preferably, the angiogenesis occurs in a human subject.

Preferably, the angiogenesis is associated with a disease or condition in a human or an animal subject that is due to, or associated with, undesired or uncontrolled angiogenesis. More preferably, the angiogensis is associated with one or more of the following diseases or conditions in a human or animal: the growth or solid tumours; angiofibroma; corneal neovascularisation; retinavchoroidal neovascularization; arteriovenous malformations; arthritis, including rheumatoid arthritis, lupus and other connective tissue disorders; Osler-Weber syndrome; atherosclerotic plaques; psoriasis; pyogenic granuloma; retrolental fibroplasias; scleroderma; granulations, hemangioma; trachoma; hemophilic joints; vascular adhesions and hypertrophic scars; diseases associated with chronic inflammation including sarcoidosis and inflammatory bowel diseases such as Crohn's disease and ulcerative colitis. More preferably, the angiogenesis is associated with corneal neovascularisation, retinal neovascularisation or choroidal neovascularisation. Most preferably, the angiogenesis is associated with corneal neovascularisation.

Diseases associated with corneal neovascularization include diabetic retinopathy, retinopathy of prematurity, corneal graft rejection, neovascular glaucoma and retrolental fibroplasia, epidemic keratoconjunctivitis, Vitamin A deficiency, contact lens overwear, atopic keratitis, superior limbic keratitis, pterygium keratitis sicca, Siogrens Syndrome, acne rosacea, phylectenulosis, syphilis, mycobacteria infections, lipid degeneration, chemical burns, bacterial ulcers, fungal ulcers, herpes simplex infections, herpes zoster infections, protozoan infections, Kaposi's sarcoma, Mooren's ulcer, Terrien's marginal degeneration, mariginal keratolysis, trauma, rheumatoid arthritis, systemic lupus, polyaneritis, Wegener's sarcoidosis, scleritis, Stevens-Johnson disease, pemphigoid, radial keratotomy.

Diseases associated with retinavchoroidal neovascularization include diabetic retinopathy, macular degeneration, sickle cell anemia, sarcoid, syphilis, pseudoxanthoma elasticum, Paget's disease, vein occlusion, artery occlusion, carotid obstructive disease, chronic uveitistvitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardts disease, pars planitis, chronic retinal detachment, hyperviscosity syndromes, toxoplasmosis, trauma and post-laser complications. Other diseases include, but are not limited to, diseases associated with rubeosis and diseases caused by the abnormal proliferation of fibrovascular or fibrous tissue including all forms of proliferative vitreoretinopathy.

The biological system may be any biological system in which angiogenesis is occurring or in which angiogenesis may occur. Preferably, the biological system is a human or animal subject in which angiogenesis is occurring. More preferably, the biological system is a human or animal subject in which angiogenesis is associated with a disease or condition that is due to undesired or uncontrolled angiogenesis. Most preferably, the biological system is a human or animal subject suffering from one or more of the following diseases or conditions associated with undesired or uncontrolled angiogenesis: angiogenesis associated with solid tumours; angiofibroma; corneal neovascularisation; retinal/choroidal neovascularization; arteriovenous malformations; arthritis, including rheumatoid arthritis, lupus and other connective tissue disorders; Osler-Weber syndrome; atherosclerotic plaques; psoriasis; pyogenic granuloma; retrolental fibroplasias; scleroderma; granulations, hemangioma; trachoma; hemophilic joints; vascular adhesions and hypertrophic scars; diseases associated with chronic inflammation including sarcoidosis and inflammatory bowel diseases such as Crohn's disease and ulcerative colitis.

The effective amount of allyl-substituted fatty acid to be administered is not particularly limited, so long as it is within such an amount and in such a form that generally exhibits a pharmacologically useful or therapeutic effect.

In this regard, an effective amount of the alkyl-substituted fatty acid may be appropriately chosen, depending upon the amount of the composition containing the alkyl-substituted fatty acid, the extent of angiogenesis to be inhibited, the age and body weight of the subject, the frequency of administration, and the presence of other active agents.

Preferably, the effective amount of alkyl-substituted fatty acid administered results in a concentration of the compound at the desired site of action in the biological system is in the range from 50 nM to 5 mM. More preferably, the effective amount of alkyl-substituted fatty acid administered results in a concentration of the compound at the desired site of action in the biological system is in the range from 50 nM to 1 mM. Most preferably, the effective amount of alkyl-substituted fatty acid administered results in a concentration of the compound at the desired site of action in the biological system is in the range from 25 μM to 500 μM.

In the case of topical administration of the alkyl-substituted fatty acid, the effective amount of the alkyl-substituted fatty acid applied topically to a desired site is preferably in the range from 25 nmol to 200 μmol.

The administration of alkyl-substituted fatty acid may be within any time suitable to produce the desired effect of inhibiting angiogenesis in the biological system. In a human or animal subject, the alkyl-substituted fatty acid may be administered orally, parenterally, topically or by any other suitable means, and therefore transit time of the drug must be taken into account.

The administration of the alkyl-substituted fatty acid may further include the administration of an immunosuppressant. Preferably, the immunosuppressant is an agent that binds to an immunophilin. More preferably, the immunosuppressant is cyclosporin A, rapamycin or FK506. Most preferably, the immunosuppressant is rapamycin.

In a preferred form, the present invention provides a method of inhibiting angiogenesis in a biological system, the method including the step of administering to the biological system an effective amount of rapamycin and 12-methyltetradecanoic acid, 13-methyltetradecanoic acid, 14-methylpentadecanoic acid, 17-methyloctadecanoic acid, 16-methylheptadecanoic acid, 10-methyidodecanoic acid, or any combination of these fatty acids.

In another preferred form, the present invention provides a method of inhibiting angiogenesis in a biological system, the method including the step of administering to-the biological system an effective amount of cyclosporin A and 12-methyltetradecanoic acid, 13-methyltetradecanoic acid, 14-methylpentadecanoic acid, 17-methyloctadecanoic acid, 16-methylheptadecanoic acid, 10-methyldodecanoic acid, or any combination of these fatty acids.

In this regard, an effective amount of the immunosuppressant may be appropriately chosen, depending upon the amount of the composition containing the immunosuppressant and the alkyl-substituted fatty acid, the extent of angiogenesis to be inhibited, age and body weight of the subject, and frequency of administration.

In the case of administration of cyclosporin A, preferably this agent is administered so that the concentration at the desired site of action in the biological system is in the range from 10 nM to 2 μM. More preferably, cyclosporin A is administered so that the concentration at the desired site of action in the biological system is in the range from 10 nM to 100 nM.

In the case of administration of rapamycin, preferably this agent is administered so that the concentration at the desired site of action in the biological system is in the range from 0.1 nM to 30 nM. More preferably, rapamycin is administered so that the concentration at the desired site of action in the biological system is in the range from 0.1 nM to 10 nM.

The administration of immunosuppressant may be within any time suitable to produce the desired effect of inhibiting angiogenesis in the biological system in conjunction with the alkyl-substituted fatty acid. In a human or animal subject the immunosuppressant may be administered orally, parenterally or by any other suitable means and therefore transit time of the drug must be taken into account. The administration of the immunosuppressant may occur at the same time and in the same manner as the administration of the alkyl-substituted fatty acid. Alternatively, the administration of the immunosuppressant may be separate to the administration of the alkyl-substituted fatty acid, and occur at a pharmacologically appropriate time before or after administration of the alkyl-substituted fatty acid.

The administration of the immunosuppressant may also include the use of one or more pharmaceutically acceptable additives, including pharmaceutically acceptable salts, amino acids, polypeptides, polymers, solvents, buffers, excipients and bulking agents.

The administration of the alkyl-substituted fatty acid may further include the administration of an anti-angiogenic agent, including anti-VEGF antibodies, including humanized and chimeric antibodies, anti-VEGF aptamers and antisense oligonucleotides, angiostatin, endostatin, interferons, interleukin 1, interleukin 12, retinoic acid, and tissue inhibitors of metalloproteinase-1 and -2.

The inhibition of angiogenesis in the biological system may be determined by a suitable method known in the art, such as delayed appearance of neovascular structures, slowed development of neovascular structures, decreased occurrence of neovascular structures, slowed or decreased severity of angiogenesis-dependent disease effects, arrested angiogenic growth, or regression of previous angiogenic growth.

Determination of the ability of the alkyl-substituted fatty acid to inhibit angiogenesis may be by any suitable assay of measuring angiogenesis that is well known in the art. For example, a chicken chorioallantoic membrane (CAM) assay or a corneal neovascularization model may be performed. The ability of a test alkyl-substituted fatty acid to inhibit angiogenesis may be determined by the extent of inhibition of angiogenesis in the chicken embryo or the extent of inhibition of angiogenesis in a corneal neovascularization model.

For example, the ability of an alkyl-substituted fatty acid (ie the test fatty acid) to inhibit angiogenesis in a chicken chorioallantoic membrane assay may be tested by contacting the chorioallantoic membrane with the alkyl-substituted fatty acid applied to a methyl cellulose disc. For the corneal neovascularization model, the alkyl-substituted fatty acid may be applied as a topical composition containing the alkyl-substituted fatty acid to the cornea, the cornea being scratched and inoculated with pseudomonas aeruginosa to induce neovascularisation.

Another method to study angiogenesis is the subcutaneous implantation of various artificial sponges (i.e. polyvinyl alcohol, gelatin) in animals. The alkyl-substituted fatty acid to be evaluated may be injected directly into the sponges, which are placed in the center of the sponge. Neovasculanzation of the sponges is assessed either histologically, morphometrically (vascular density), biochemically (hemoglobin content) or by measuring the blood flow rate in the vasculature of the sponge using a radioactive tracer.

Numerous animal tumor models have-also been developed to test the anti-angiogenic activity of test compounds. In many cases, tumor cells are engrafted subcutaneously and tumor size is determined at regular time intervals. Frequently used tumor cells include C6 rat glioma, B16BL6 melanoma, LLC, and Walker 256 carcinoma.

As will be appreciated, in determining the ability of a test fatty acid to inhibit the angiogenesis, the test fatty acid will be delivered at a concentration and in form that are suitable to the particular physical and chemical characteristics of the test fatty acid.

In a preferred form, the present invention also provides a method of inhibiting neovascularisation of a cornea, the method including the step of administering to the cornea an effective amount of an-alkyl-substituted fatty acid, wherein the alkyl-substituted fatty acid is capable of inhibiting neovascularisation of the cornea and the alkyl-substituted fatty acid has the following chemical formula: 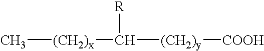
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH=CH group or a C≡C group, and x+y is between 2 and 46.

Examples of neovascularisabon of the cornea include neovascularisation associated with wearing contact lenses, trauma of the cornea, burns, bacterial infections of the cornea such as infections caused by chiamydia, staphylococcus, or pseudomonas (eg pseudomonas aenrginosa), viral infections such as infections caused by herpes simplex and herpes zoster, protozoan infections, immunological diseases, and degenerative disorders.

The alkyl-substituted fatty acid may be administered by a suitable method known in the art, including topical administration to the cornea. For example, the alkyl-substituted fatty acid may be prepared as an emulsion in unpreserved paraffin and lanolin ophthalmic ointment base, and the composition applied topically to the cornea. In this case, the effective amount of the alkyl-substituted fatty acid applied topically is preferably in the range from 25 nmol to 200 μmol.

In another preferred form, the present invention provides a method of inhibiting neovascularisation of a cornea, the method including the step of administering to the cornea an effective amount of 12-methyltetradecanoic acid, 13-methyltetradecanoic acid, 14-methylpentadecanoic acid, 17-methyloctadecanoic acid, 16-methylheptadecanoic acid, 10methyldodecanoic acid, or any combination of these alkyl-substituted fatty acids.

The present invention also provides a method of reducing the amount of an agent administered to a biological system to achieve a desired level of inhibition of endothelial cell proliferation, the method including the step of administering to the biological system an effective amount of an alkyl-subsututed fatty acid, wherein the alkyl-substituted fatty acid has the following chemical formula: 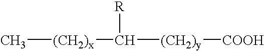
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

In this regard, it has also surprisingly found that the amount of an agent administered to a biological system to inhibit endothelial cell proliferation may be reduced by also administering an alkyl-substituted fatty acid. For example, the amount of cyclosporin A or rapamycin required to achieve a desired level of inhibition of endothelial cell proliferation is reduced in the presence of 12-methyltetradecanoic acid.

The endothelial cell is any endothelial cell, including an endothelial cell that is undergoing proliferation in response to one or more angiogenic stimuli, or an endothelial cell that has the capacity to undergo proliferation in response to one or more angiogenic stimuli. Preferably, the endothelial cell is a human or animal endothelial cell. Most preferably, the endothelial cell is a human endothelial cell.

Preferably, the endothelial cell is undergoing proliferation associated with a disease or condition in a human or an animal subject that is associated with undesired or uncontrolled angiogenesis. More preferably, the endothelial cell is undergoing proliferation associated with one or more of the following diseases or conditions: angiogenesis associated with solid tumours; angiofibroma; corneal neovascularisation; retinal/choroidal neovascularization; arteriovenous malformations; arthritis, including rheumatoid arthritis, lupus and other connective tissue disorders; Osler-Weber syndrome; atherosclerotic plaques; psoriasis; pyogenic granuloma; retrolental fibroplasias; scleroderma; granulations, hemangioma; trachoma; hemophilic joints; vascular adhesions and hypertrophic scars; diseases associated with chronic inflammation including sarcoidosis and inflammatory bowel diseases such as Crohn's disease and ulcerative colitis. More preferably, the angiogenesis is associated with corneal neovascularisation, retinal neovascularisation or choroidal neovascularisation. Most preferably, the angiogenesis is associated with corneal neovascularisation.

The biological system is any system that includes endothelial cells that have the capacity to proliferate, or any system that includes endothelial cells that are proliferating. Preferably, the biological system is a human or animal subject that includes endothelial cells that have the capacity to proliferate, or endothelial cells that are proliferating. More preferably, the biological system is a human or animal subject that includes the proliferation of endothelial cells associated with a disease or condition that is due to undesired or uncontrolled angiogenesis. More preferably, the biological system is a human or animal subject suffering from a disease or condition involving the proliferation of endothelial cells. Most preferably, the biological system is a human or animal subject suffering from one or more of the following diseases or conditions associated with the proliferation of endothelial cells: angiogpnesis associated with solid tumours; angiofibroma; corneal neovascularisation; retinavchoroidal neovascularization; arteriovenous malformations; arthritis, including rheumatoid arthritis, lupus and other connective tissue disorders; Osler-Weber syndrome; atherosclerotic plaques; psoriasis; pyogenic granuloma; retrolental fibroplasias; scleroderma; granulations, henagioma; trachoma; hemophilic joints; vascular adhesions and hypertrophic scars; diseases associated with chronic inflammation including sarcoidosis and inflammatory bowel diseases such as Crohn's disease and ulcerative colitis.

The effective amount of the alkyl-substituted fatty acid to be administered is not particularly limited, so long as it is within such an amount and in such a form that generally exhibits a pharmacologically useful effect to reduce the amount of agent necessary to achieve a desired level of inhibition of endothelial cell proliferation in the biological system.

Preferably, the effective amount of alkyl-substituted fatty acid administered results in a concentration of the compound at the desired site of action in the biological system is in the range from 50 nM to 5 mM. More preferably, the effective amount of alkyl-substituted fatty acid administered results in a concentration of the compound at the desired site of action in the biological system is in the range from 50 nM to 1 mM. Most preferably, the effective amount of alkyl-substituted fatty acid administered results in a concentration of the compound at the desired site of action in the biological system in the range from 25 μM to 500 μM.

The administration of the alkyl-substituted fatty acid may be within any time suitable to produce the desired effect of reducing the amount of an agent administered to a biological system necessary to achieve a desired level of inhibition of endothelial cell proliferation in the biological system. In a human or animal subject, the alkyl-substituted fatty acid may be administered orally, parenterally, topically or by any other suitable means, and therefore transit time of the drug must be taken into account.

Examples of agents capable of inhibiting endothelial cell proliferation include rapamycin, cyclosporin A, RTNP-470-(a fumagillin derivative), squalamine, combretastatin, endostatin, penicillamine, famesyl transferase inhibitor, L-778,123 (Merck), SCH66336 (Schering-Plough), and R115777 (Janssen). Preferably, the agent is rapamycin or cyclosporin A. Most preferably, the agent is rapamycin.

For example, 1 nM rapamycin inhibits the proliferation of HUVECs in vftro after 24 hours by approximately 80%. The same level of inhibition (87%) of proliferation may also be achieved in these cells with only 0.1 nM rapamycin, if 100 μM 12-methyltetradecanoic acid is also present. Thus the presence of the alkyl-substituted fatty acid reduces the amount of rapamycin necessary to achieve a desired level of inhibition of endothelial cell proliferation.

In a preferred form, the present invention provides a method of reducing the amount of rapamycin and/or cyclosporin A administered to a biological system to achieve a desired level of inhibtion of endothelial cell proliferation, the method including the step of administering to the biological system an effective amount of 16-methyl heptadecanoic acid, 15-methyl heptadecanoic acid, 15-methyl hexadecanoic acid, 14-methyl hexadecanoic acid, 14-methyl pentadecanoic acid, 13-methyl pentadecanoic acid, 13-methyl tetradecanoic acid, 12-methyl tetradecanoic acid, 12-methyl tridecanoic acid, 11-methyl tridecanoic acid, 11-methyl dodecanoic acid, and 10-methyl undecanoic acid, or any combination of these alkyl-substituted fatty acids.

In this regard, the amount of the agent necessary to achieve a desired level of inhibition of endothelial cell proliferation will be empirically determined by a method known in the art, and as such will depend upon the desired level of endothelial proliferation to be inhibited, the age and body weight of the subject, and the frequency of administration.

In the case of administration of rapamycin, preferably this agent is administered so that the concentration of the compound at the desired site of action in the biological system is in the range from 0.1 nM to 30 nM. More preferably, rapamycin is administered so that the concentration of the compound at the desired site of action in the biological system is in the range from 0.1 nM to 10 nM.

The administration of the agent necessary to achieve a desired level of inhibition of endothelial cell proliferation will be in a suitable form and within a suitable time to produce the desired effect of inhibiting the proliferation of endothelial cells to the desired level.

The alkyl-substituted fatty acid may be administered orally, parenterally, topically or by any other suitable means and therefore transit time of the drug must be taken into account. The administration of the alkyl-substituted fatty acid may occur at the same time and in the same manner as the administration of the agent capable of inhibiting endothelial cell proliferation in the biological system. Alternatively, the administration of the alkyl-substituted fatty acid may be separate to the administration of the agent capable of inhibiting endothelial cell proliferation in the biological system, and occur at a pharmacologically appropriate time before or after administration of the agent.

The present invention also provides a method of reducing the amount of an anti-angiogenic agent administered to a biological system to achieve a desired level of inhibition of angiogenesis, the method including the step of administering to the biological system an effective amount of an alkyl-substituted fatty acid, wherein the alkyl-substituted fatty acid has the following chemical formula: 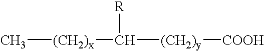
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

The angiogenesis may be any angiogenesis occurring in a biological system. Preferably, the angiogenesis occurs in an animal or human subject. Most preferably, the angiogenesis occurs in a human subject.

Preferably, the angiogenesis is associated with a disease or condition in a human or an animal that is due to, or associated with, uncontrolled or undesired angiogenesis. More preferably, the angiogensis is associated with one or more of the following diseases or conditions in a human or animal: the growth or solid tumours; angiofibroma; corneal neovascularisation; retinal/choroidal neovascularization; arteriovenous malformations; arthritis, including rheumatoid arthritis, lupus and other connective tissue disorders; Osler-Weber syndrome; atherosclerotic plaques; psoriasis; pyogenic granuloma; retrolental fibroplasias; scleroderma; granulations, hemangioma; trachoma; hemophilic joints; vascular adhesions and hypertrophic scars; diseases associated with chronic inflammation including sarcoidosis and inflammatory bowel diseases such as Crohn's disease and ulcerative colitis._More preferably, the angiogenesis is associated with corneal neovascularisation, retinal neovascularisation or choroidal neovascularisation. Most preferably, the angiogenesis is associated with corneal neovascularisation.

The biological system may be any biological system in which angiogenesis is occurring or in which angiogenesis may occur. Preferably, the biological system is a human or animal subject in which angiogenesis is occurring. More preferably, the biological system is a human or animal subject in which angiogenesis is associated with a disease or condition that is due to undesired angiogenesis. Most preferably, the biological system is a human or animal subject suffering from one or more of the following diseases or conditions associated with undesired or uncontrolled angiogenesis: angiogenesis associated with solid tumours; angiofibroma; corneal neovascularisation; retinal/choroidal neovascularization; arteriovenous malformations; arthritis, including rheumatoid arthritis, lupus and other connective tissue disorders; Osler-Weber syndrome; atherosclerotic plaques; psoriasis; pyogenic granuloma; retrolental fibroplasias; scleroderma; granulations, hemangioma; trachoma; hemophilic joints; vascular adhesions and hypertrophic scars; diseases associated with chronic inflammation including sarcoidosis and inflammatory bowel diseases such as Crohn's disease and ulcerative colitis.

The effective amount of alkyl-substituted fatty acid to be administered is not particularly limited, so long as it is within such an amount that generally exhibits a pharmacologically useful effect to reduce the amount of agent necessary to achieve a desired level of inhibition of angiogenesis in the biological system.

Preferably, the effective amount of alkyl-substituted fatty acid administered results in a concentration of the compound at the desired site of action in the range from 50 nM to 5 mM. More preferably, the effective amount of alkyl-substituted fatty acid administered results in a concentration of the compound at the desired site of action in the range from 50 nM to 1 mM. Most preferably, the effective amount of alkyl-substituted fatty acid administered results in a concentration of the compound at the desired site of action in the range from 25 μM to 500 μM.

The administration of the alkyl-substituted fatty acid may be within any time suitable to produce the desired effect of reducing the amount of an agent administered to a biological system necessary to achieve a desired level of inhibition of angiogenesis in the biological system. In a human or animal subject, the alkyl-substituted fatty acid may be administered orally, parenterally, topically or by any other suitable means, and therefore transit time of the drug must be taken into account.

Examples of anti-angiogenic agents include anti-VEGF antibodies, including humanized and chimeric antibodies, anti-VEGF aptamers and antisense oligonucleotides, angiostatin, endostatin, interferons, interleukin 1, interleukin 12, retinoic acid, and tissue inhibitors of metalloproteinase-1 and -2.

In this regard, the amount of the anti-angiogenic agent necessary to achieve a desired level of inhibition of angiogenesis will be empirically determined by a method known in the art, and as such will depend upon the desired level of angiogenesis to be inhibited, the age and body weight of the subject, and the frequency of administration.

The administration of the anti-angiogenic agent will be in a suitable form and within a suitable time to produce the desired effect of inhibiting angiogenesis to the desired level.

The alkyl-substituted fatty acid may be administered orally, parenterally, topically or by any other suitable means and therefore transit time of the drug must be taken into account. The administration of the alkyl-substituted fatty acid may occur at the same time and in the same manner as the administration of the anti-angiogenic agent. Alternatively, the administration of the alky-substituted fatty acid may be separate to the administration of the anti-angiogenic agent, and occur at a pharmacologically appropriate time before or after administration of the agent.

The present invention further provides a pharmaceutical composition including an alkyl-substituted fatty acid, wherein the alkyl-substituted fatty acid is capable of inhibiting endothelial cell proliferation and/or angiogenesis and the alkyl-substituted fatty acid has the following chemical formula: 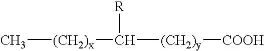
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

Preferably, the alkyl-substituted fatty acid is 18-methylnonadecanoic acid, 17-methyloctadecanoic acid, 10-methyloctadecanoic acid, 1 6-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyldodecanoic acid, or any combination of these alkyl-substituted fatty acids.

The amount of the alkyl-substituted fatty acid to be used in the pharmaceutical composition is not particularly limited, so long as it is within such an amount that generally will exhibit a pharmacologically therapeutic or useful effect when the composition is administered to a subject.

The amount of the alkyl-substituted fatty acid in the pharmaceutical composition may be appropriately chosen, depending upon the extent of angiogenesis or endothelial cell proliferation to be inhibited, the age and body weight of the subject, and the frequency of administration.

Preferably, the amount of the alkyl-substituted fatty acid in the pharmaceutical composition will be such that when the composition is administered to a subject the concentration of the compound at the desired site of action is in the range from 50 nM to 5 mM. More preferably, the amount of the alkyl-substituted fatty acid in the pharmaceutical composition will be such that when the composition is administered to a subject the concentration of the compound at the desired site of action is in the range from 50 nM to 1 mM. Most preferably, the amount of the alkyl-substituted fatty acid in the pharmaceutical composition will be such that when the composition is administered to a subject the concentration of the compound at the desired site of action is in the range from 25 μM to 500 μM.

In the case of topical administration of the alkyl-substituted fatty acid, the effective amount of the alkyl-substituted fatty acid applied topically to a desired site is preferably in the range from 25 nmol to 200 μmol.

The pharmaceutical composition may also include the use of one or more pharmaceutically acceptable additives, including pharmaceutically acceptable salts, amino acids, polypeptides, polymers, solvents, buffers, excipients and bulking agents, taking into account the physical and chemical properties of the alkyl-substituted fatty acid.

For example, the alkyl-substituted fatty acid can be prepared into a variety of pharmaceutical preparations in the form of, e.g., an aqueous solution, an oily preparation, a fatty emulsion, an emulsion, a gel, etc., for administration as intramuscular or subcutaneous injection or as injection to the organ, or as an embedded preparation or as a transmucosal preparation through nasal cavity, rectum, uterus, vagina, lung, etc. The composition of the present invention can also be administered in the form of oral preparations (for example solid preparations such as tablets, capsules, granules or powders; liquid preparations such as syrup, emulsions or suspensions). Compositions containing the alkyl-substituted fatty acid may also contain a preservative, stabiliser, dispersing agent, pH controller or isotonic agent. Examples of suitable preservatives are glycerin, propylene glycol, phenol or benzyl alcohol. Examples of suitable stabilisers are dextran, gelatin, α-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

When administered orally, the composition will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Molded tablets may be made by molding in a suitable machine, a mixture of the powdered active ingredient and a suitable carrier moistened with an inert liquid diluent.

The pharmaceutical compositions may utilize controlled release or sustained release technology. To further increase the sustained release effect, the composition may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; altematively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose, (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, the alkyl-substituted fatty acid may be incorporated into a hydrophobic polymer matrix for controlled release over a period of days. The composition of the invention may then be molded into a solid implant, or externally applied patch, suitable for providing efficacious concentrations of the alkyl-substituted fatty acid over a prolonged period of time without the need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time release characteristics and release kinetics. The composition may then be molded into a solid implant suitable for providing efficacious concentrations of the alkyl-substituted fatty acid over a prolonged period of time without the need for frequent re-dosing. The alkyl-substituted fatty acid can be incorporated into the biodegradable polymer or polymer mixture in any suitable manner known to one of ordinary skill in the art and may form a homogeneous matrix with the biodegradable polymer, or may be encapsulated in some way within the polymer, or may be molded into a solid implant.

In another form, the present invention provides the use of an alkyl-substituted fatty acid for the preparation of a medicament for inhibiting endothelial cell proliferation and/or inhibiting angiogenesis, wherein the alkyl-substituted fatty acid has the following chemical formula: 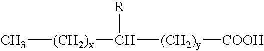
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂CH₂ group in (CH₂)ₓ and/or (CH₂)_{y} is replaced with a CH═CH group or a C≡C group, and x+y is between 2 and 46.

The pharmaceutical composition may further include an immunosuppressant. Preferably, the immunosuppressant is an agent that binds to an immunophilin. More preferably, the immunosuppressant is cyclosporin A, rapamycin or FK506. Most preferably, the immunosuppressant is rapamycin.

Accordingly, in a preferred form, the present invention also provides a pharmaceutical composition including an alkyl-substituted fatty acid and immunosuppressant, wherein the alkyl-substituted fatty acid has the following chemical formula: 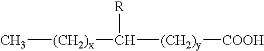
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

A dose of the immunosuppressant in the composition may be appropriately chosen, depending upon the amount of the composition containing the immunosuppressant and the alkyl-substituted fatty acid, the extent of angiogenesis to be inhibited, the age and body weight of the subject, and the frequency of administration.

In the case of the pharmaceutical composition containing cyclosporin A, preferably this agent is present in the composition such that when administered to a subject the concentration of the agent at the site of action is in the range from 10 nM to 2 μM. More preferably, this agent is present in the composition such that when administered to a subject the concentration of the agent at the site of action is in the range from 10 nM to 100 nM.

In the case of the pharmaceutical composition containing rapamycin, preferably this agent is present in the composition such that when administered to a subject the concentration of the agent at the site of action is in the range from 0.1 nM to 30 nM. More preferably, this agent is present in the composition such that when administered to a subject the concentration of the agent at the site of action is in the range from 0.1 nM to 10 nM.

To facilitate the administration of the immunosuppressant, the composition may also include the use of one or more pharmaceutically acceptable additives, including pharmaceutically acceptable salts, amino acids, polypeptides, polymers, solvents, buffers, excipients and bulking agents, or any other additive that aids in the control of the release of the alkyl-substituted fatty acid or immunosuppressant agent, or aids in the delivery of the alkyl-substituted fatty acid or immunosuppressant to a subject.

In another preferred form, the present invention provides a pharmaceutical composition including an alkyl-substituted fatty acid, wherein the alkyl-substituted fatty acid is capable of inhibiting corneal neovascularisation and the alkyl-substituted fatty acid has the following chemical formula: 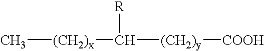
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

Preferably, the alkyl-substituted fatty acid is 18-methylnonadecanoic acid, 17-methyloctadecanoic acid, 10-methyloctadecanoic acid, 16-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyldodecanoic acid, or any combination of these alkyl-substituted fatty acids.

In another form, the present invention provides the use of an alkyl-substituted fatty acid and an immunosuppressant for the preparation of a medicament for inhibiting endothelial cell proliferation and/or inhibiting angiogenesis, wherein the alkyl-substituted fatty acid has the following chemical formula: 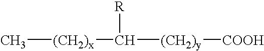
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

### DESCRIPTION OF THE PREFERRED EMBODIMENTS

Reference will now be made to experiments that embody the above general principles of the present invention. However, it is to be understood that the following description is not to limit the generality of the above description.

### EXAMPLE 1

Preparation of 12-methyltetradecanoic acid (12-MTA) and other alkyl-substituted fatty acids

12-methyltetradecanoic acid and other alkyl-substituted fatty acids were obtained from Sigma Chemicals.

Due to the poor aqueous solubility of 12-methyltetradecanoic acid, the compound was dissolved in 95% ethanol at a stock concentration of 100 mM. Further dilutions were also performed in 95% ethanol and working concentrations for experiments in the range from 25 μM to 800 μM were diluted in culture medium with a final ethanol concentration of less than 0.8%. Control samples with no added agent in the culture medium contained less than 0.8% ethanol.

Other alkyl-substituted fatty acids were prepared in a similar manner.

### EXAMPLE 2

HUVEC Proliferation Assay

Human umbilical vein endothelial cells (HUVEC) were seeded in 96-well flat bottomed tissue culture plates at a density of 2.5-5×10⁴ cells/well and treated with various dilutions of agents. Cells were cultured in RPMI medium containing 20% FCS, Penicillin/Streptomycin in a 5% CO₂ atmosphere at 37° C. After 24 to 48 hours of incubation, cells were pulsed with 1 μCi of tritiated thymidine for 6 hours. The pulsed cells were trypsinised to detach from the wells and then harvested in a TOMTEC Cell Harvester onto glass fibre filters, which were dried and immersed in scintillation fluid and counted in a Wallac Microbeta scintillation counter. The results were reported as mean cpm±SD.

### EXAMPLE 3

Effect of 12-MTA on HUVEC Proliferation

The tritiated thymidine uptake assay demonstrated that HUVEC proliferation was inhibited in a dose response manner at increasing concentrations of 12-MTA (Table 1). Inhibition was expressed as a percentage of control cells that had no added agent.

At the concentration of 800 μM, microscopic examination of the cells demonstrated the appearance of apoptotic cells. However, at concentrations between 50 to 400 μM, cells demonstrated good viability but thymidine incorporation into the DNA was inhibited, demonstrating the inhibition of proliferation of the HUVECS by 12-MTA. The inhibition ranged from 99% at 800 AM 12-MTA to 13% inhibition at 50 μM 12-MTA

TABLE 1
Dose response inhibition of HUVEC proliferation with 12-MTA

| MTA | Average | STDEV | Sample 1 | Sample 2 | Sample 3 | % Inhibition |
|---|---|---|---|---|---|---|
| 800 μM | 9.0 | 2.0 | 11 | 7 | 9 | 99 |
| 400 μM | 12104.7 | 1998.0 | 12558 | 13837 | 9919 | 44.1 |
| 200 μM | 15707.7 | 2657.5 | 18491 | 15435 | 13197 | 27.5 |
| 100 μM | 17593.0 | 2518.8 | 20367 | 16963 | 15449 | 18.7 |
| 50 μM | 18781.3 | 1468.8 | 19272 | 19942 | 17130 | 13.3 |
| EtOH | 21652.0 | 3068.5 | 23255 | 23587 | 18114 | 0 |

### EXAMPLE 4

Effect of 12-MTA in Comparison to Other Agents on HUVEC Proliferation

The inhibition of HUVEC proliferation was used to compare the effects of 12-MTA with cyclosponin A and rapamycin, both of which have antiangiogenic properties. The concentrations of cyclosporin A (10 nM and 100 nM) and rapamycin (0.1 nM and 1 nM) were based on concentrations that were known to inhibit lymphocyte proliferation based on previous studies conducted in the laboratory. The representative data from three different experiments is shown in Table 2.

TABLE 2
Synergistic inhibitory effects on HUVEC proliferation of
12-MTA in combination with cyclosporin A and rapamycin

| | % INHIBITION |
|---|---|
| 0.1 nM rapamycin | 72.8 |
| 1 nM rapamycin | 77.9 |
| 10 nM CsA | −26.6 |
| 100 nM CsA | −47.5 |
| 100 μM 12-MTA | 24.8 |
| 0.1 nM rap/100 μM MTA | 87 |
| 1 nM rap/100 μM MTA | 93 |
| 10 nM CsA/100 μM MTA | 69.7 |
| 100 nM CsA/100 μM MTA | 68 |
| 200 μM 12-MTA | 30.2 |
| 0.1 nM rap/200 μM MTA | 92 |
| 10 nM CsA/200 μM MTA | 64.4 |
| EtOH alone | 0.0 |

Table 2 shows that HUVEC proliferation was inhibited by 73% and 78% with 0.1 nM and 1 nM rapamycin, respectively. However, cyclosporin A showed stimulation of HUVEC proliferation at both 10 nM and 100 nM concentrations.

Suboptimal inhibitory concentrations of both 12-MTA and cyclosporin A or rapamycin were also combined and added to HUVEC cultures for a period of 24 hours and then assessed for proliferation. As shown in Table 2, synergistic inhibitory effects were observed with combinations of 10 nM and 100 nM cyclosporin A, respectively, with 100 μM 12-MTA. However, due to the strong inhibitory effect of rapamycin alone only additive effects with 12-MTA were observed in these experiments.

This data also demonstrates that the levels of cyclosporin A or rapamycin necessary to inhibit the proliferation of HUVECs in vitro after 24 hours may be lowered if 12-methyltetradecanoic acid is also present. Thus the presence of the alkyl-substituted fatty acid reduces the amount of these agents necessary to achieve a desired level of inhibition of endothelial cell proliferation.

### EXAMPLE 5

Effect of Other Alkyl-substituted Fatty Acids on HUVEC Proliferation

The tritiated thymidine uptake assay demonstrated that HUVEC proliferation was also inhibited by the following alkyl substituted fatty acids at 400 μM concentration: 16-methyl heptadecanoic acid, 15-methyl heptadecanoic acid, 15-methyl hexadecanoic acid, 14-methyl hexadecanoic acid, 14-methyl pentadecanoic acid, 13-methyl pentadecanoic acid, 13-methyl tetradecanoic acid, 12-methyl tetradecanoic acid, 12-methyl tridecanoic acid, 11-methyl tridecanoic acid, 11-methyl dodecanoic acid, and 10-methyl undecanoic acid.

The data is shown in Table 3. Inhibition was expressed as a percentage of control cells that had no added agent.

TABLE 3
Percentage inhibition of HUVEC proliferation with various
alkyl-substituted fatty acids at 400 μM concentration

| | |
|---|---|
| 16-methyl heptadecanoic acid | 19% |
| 15-methyl heptadecanoic acid | 49% |
| 15-methyl hexadecanoic acid | 63% |
| 14-methyl hexadecanoic acid | 41% |
| 14-methyl pentadecanoic acid | 7% |
| 13-methyl pentadecanoic acid | 21% |
| 13-methyl tetradecanoic acid | 19% |
| 12-methyl tetradecanoic acid | 32% |
| 12-methyl tridecanoic acid | 35% |
| 11-methyl tridecanoic acid | 42% |
| 11-methyl dodecanoic acid | 83% |
| 10-methyl undecanoic acid | 84% |

### EXAMPLE 6

Chicken Chonoallantoic Membrane (CAM) Assay for Angiogenesis

Fertilised chicken eggs (HiChick Breeding Co, Kapunda, South Australia) were incubated for three days at 38° C. On Day 3 the embryos were cracked out of the egg and into a cup made of plastic piping, with plastic film stretched over the top to form a hammock for the egg to be suspended in. Two ml of DMEM containing penicillin and streptomycin was added to each cup prior to the egg being added. A petri dish on the top maintained sterility. Incubation continued in a humidified 37° C. incubator.

On Day 4 the chorioallantoic membrane (CAM) begins to grow, and pictures were taken of each embryo at ×5 to measure the CAM area using image analysis software (Video Pro 32, Leading Edge Pty Ltd, South Australia).

Embryos were then grouped according to their CAM area, with a control embryo in each for comparison. Grouping is critical as in these early developmental stages changes in the CAM growth are dramatic. Relatively small differences in size on Day 4 translate to large differences in the CAM on Day 5. Treatment was applied in methylcellulose discs, which were dried under vacuum overnight. The methylcellulose discs were applied to the top of the CAM, and at the beginning of treatment were at least three to four-fold bigger than the CAM area, meaning treatment covered the entire CAM surface.

On Day 5 skim milk with contrast medium was injected into the CAM. Pictures were then taken at various levels of magnification up to ×63. Quantitative measurements were made from ×5 pictures. CAM area, and vein and artery lengths were measured using image analysis (Video Pro 32, Leading Edge Pty Ltd, South Australia). Relative vessel lengths were then calculated as the total length/CAM area. Statistical analysis was made using SigmaStat and OneWay ANOVA with p<0.05 as the level of significance.

### EXAMPLE 7

Effect of 12-MTA on Angiogenesis in the CAM Assay

12-MTA was applied to the CAM in amounts ranging from 25 nmol to 500 nmol. Six different embryos were used for each amount of 12-MTA. Colchicin was used as a positive control for the inhibition of angiogenesis. The negative control (vehicle) was an ethanol solution, since 12-MTA was dissolved in ethanol.

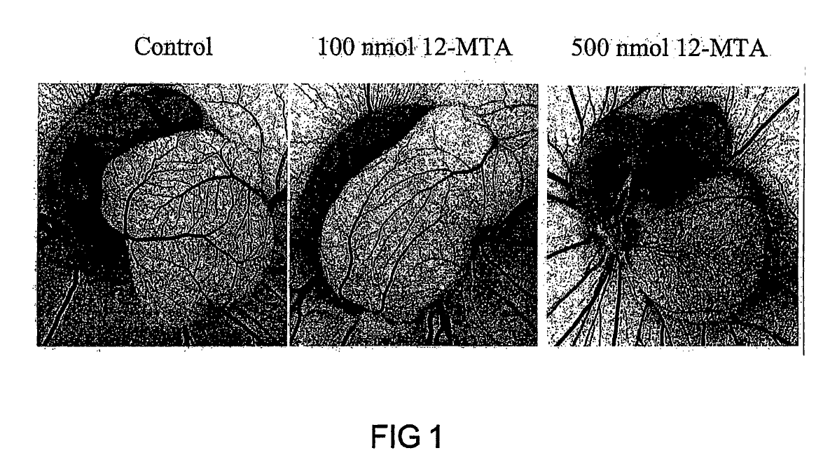

FIG. 1 shows that treatment with 500 nmol of 12-MTA yielded a reduction in the number of branching capillaries sprouting from the main vessels. In addition the vessel area is also diminished with the treatment. Similar reduction in vessel area was also observed at the 100 nmol amount. These results also demonstrate that 12-MTA was not cytotoxic to the embryo.

Quantitative measurement of the inhibitory effect of 12-MTA on angiogenesis in the CAM assay is shown in Table 4.

TABLE 4
Inhibitory effect of 12-MTA on angiogenesis in the CAM assay

| | Vehicle | 25 nmol | 50 nmol | 100 nmol |
|---|---|---|---|---|
| Vein length (%) | 100.0 ± 0.0 | 82.1 ± 22.9 | 74.4 ± 20.8 | 46.0 ± 8.7¹ |
| Artery length (%) | 100.0 ± 0.0 | 81.8 ± 11.6 | 78.8 ± 12.2 | 63.8 ± 4.0¹ |
| Total vessel length (%) | 100.0 ± 0.0 | 79.8 ± 15.0 | 74.9 ± 14.7 | 54.9 ± 4.3¹ |
| Vein Diameter (%) | 100.0 ± 0.0 | 89.3 ± 25.2 | 59.1 ± 11.2 | 45.9 ± 10.0¹ |

(% of control; n = 6 Mean ± SEM)

As can be seen, even the lowest dose of 12-MTA inhibited vein length, artery length, total vessel length and vein diameter. The extent of inhibition increased with increasing dose of 12-MTA.

### EXAMPLE 8

Effect of 10-methyloctadecanoic Acid (10-MODA) on Angiogenesis in the CAM Assay

10-MODA was applied to the CAM at various amounts. Five different embryos were used for each amount of 10-MODA and a negative control was treated with ethanol solution.

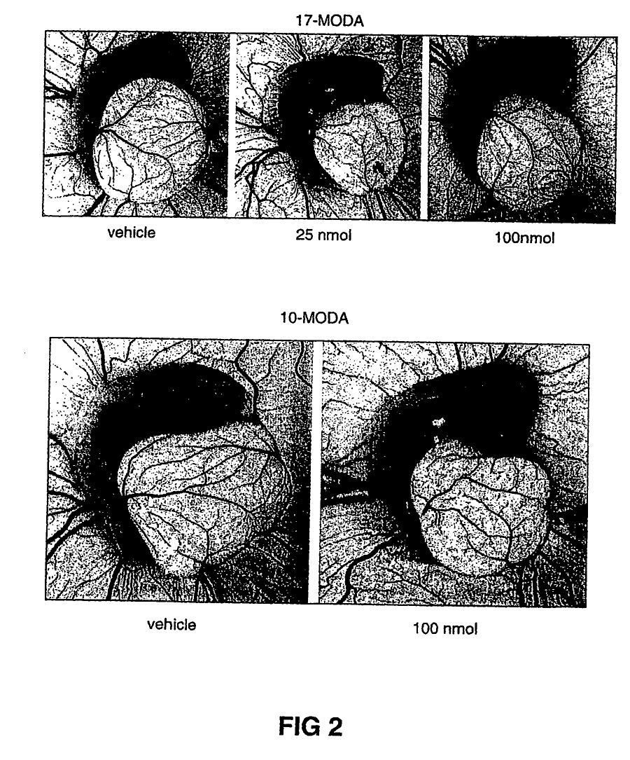

FIG. 2 shows that treatment with 100 nmol of 10-MODA yielded a reduction in the number of branching capillaries sprouting from the main vessels. In addition the vessel area is also diminished with the treatment. These results also demonstrate that 10-MODA was not cytotoxic to the embryo.

Quantitative measurement of the inhibitory effect of 10-MODA on angiogenesis in the CAM assay is shown in Table 5.

TABLE 5
Inhibitory effect of 10-MODA on angiogenesis in the CAM assay

| | Vehicle | 25 nmol | 50 nmol | 100 nmol |
|---|---|---|---|---|
| Vein length (%) | 100.0 ± 0.0 | 88.4 ± 8.8 | 85.8 ± 14.1 | 70.9 ± 22.5 |
| Artery length (%) | 100.0 ± 0.0 | 85.7 ± 6.2 | 94.3 ± 6.3 | 69.3 ± 7.5¹ |
| Total vessel length (%) | 100.0 ± 0.0 | 85.7 ± 5.4 | 88.9 ± 7.0 | 68.0 ± 11.8¹ |
| Vein Diameter (%) | 100.0 ± 0.0 | 82.9 ± 3.0 | 90.2 ± 9.8 | 76.1 ± 16.2 |

(% of control; n = 5, Mean ± SEM)

As can be seen, even the lowest dose of 10-MODA inhibited vein length, artery length, total vessel length and vein diameter. The extent of inhibition increased with increasing dose of 10-MODA.

### EXAMPLE 9

Effect of 13-methyltetradecanoic Acid (13-MTA) on Angiogenesis in the CAM Assay

13-MTA was applied to the CAM at various amounts. Five different embryos were used for each amount of 13-MTA and a negative control (vehicle) was treated with ethanol solution.

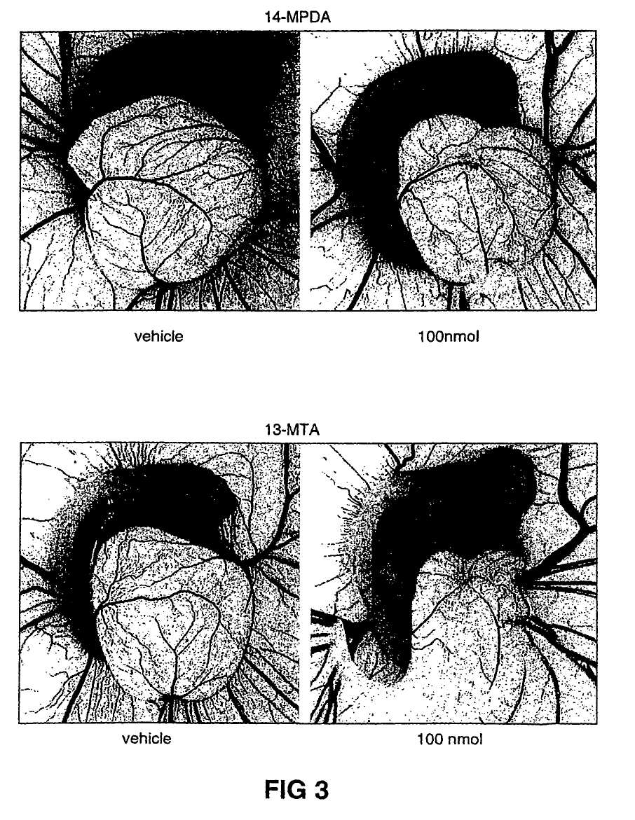

FIG. 3 shows in the bottom panel that treatment with 100 nmol of 13-MTA yielded a reduction in the number of branching capillaries sprouting from the main vessels. In addition the vessel area is also diminished with the treatment. These results also demonstrate that 13-MTA was not cytotoxic to the embryo.

Quantitative measurement of the inhibitory effect of 13-MTA on angiogenesis in the CAM assay is shown in Table 6.

TABLE 6
Inhibitory effect of 10-MODA on angiogenesis in the CAM assay

| | Vehicle | 25 nmol | 50 nmol | 100 nmol |
|---|---|---|---|---|
| CAM Increase (%) | 100.0 ± 0.0 | 101.9 ± 11.2 | 120.2 ± 25.5 | 68.2 ± 8.6 |
| Vein length (%) | 100.0 ± 0.0 | 87.2 ± 14.5 | 87.6 ± 12.1 | 44.1 ± 11.7^{1,2,3} |
| Artery length (%) | 100.0 ± 0.0 | 86.2 ± 11.2 | 92.6 ± 28.0 | 48.0 ± 12.9 |
| Total vessel length (%) | 100.0 ± 0.0 | 86.2 ± 12.1 | 89.3 ± 19.5 | 46.3 ± 12.3^{1,2,3} |
| Vein Diameter (%) | 100.0 ± 0.0 | 91.6 ± 18.0 | 82.4 ± 14.0 | 45.9 ± 10.4^{1,2,3} |

(% of control; n = 5, Mean ± SEM)

As can be seen, even the lowest dose of 13-MTA inhibited vein length, artery length, total vessel length and vein diameter. The extent of inhibition increased with increasing dose of 13-MTA.

### EXAMPLE 10

Effect of 14-methylpentadecanoic Acid (14-MPDA) on Angiogenesis in the CAM Assay

14-MPDA was applied to the CAM at various amounts. Five different embryos were used for each amount of 14-MPDA and a negative control (vehicle) was treated with ethanol solution.

FIG. 3 shows in the top panel that treatment with 100 nmol of 14-MPDA yielded a reduction in the number of branching capillaries sprouting from the main vessels. In addition the vessel area is also diminished with the treatment. These results also demonstrate that 14-MPDA was not cytotoxic to the embryo.

Quantitative measurement of the inhibitory effect of 14-MPDA on angiogenesis in the CAM assay is shown in Table 7.

TABLE 7
Inhibitory effect of 14-MPDA on angiogenesis in the CAM assay

| | Vehicle | 25 nmol | 50 nmol | 100 nmol |
|---|---|---|---|---|
| Vein length | 100.0 ± 0.0 | 89.6 ± 10.1 | 80.9 ± 14.3 | 63.3 ± 5.1¹ |
| (%) | | | | |
| Artery length | 100.0 ± 0.0 | 81.3 ± 11.4 | 81.4 ± 11.7 | 83.5 ± 9.3 |
| (%) | | | | |
| Total vessel | 100.0 ± 0.0 | 84.7 ± 8.7 | 80.9 ± 12.3 | 74.0 ± 6.6 |
| length (%) | | | | |
| Vein Diameter | 100.0 ± 0.0 | 90.8 ± 9.2 | 81.8 ± 14.1 | 76.3 ± 10.2 |
| (%) | | | | |

(% of control; n = 5, Mean ± SEM)

As can be seen, even the lowest dose of 14-MPDA inhibited vein length, artery length, total vessel length and vein diameter. The extent of inhibition increased with increasing dose of 14-MPDA.

### EXAMPLE 11

Effect of 17-methyloctadecanoic Acid (17-MODA) on Angiogenesis in the CAM Assay

17-MODA was applied to the CAM at various amounts. Six different embryos were used for each concentration of 17-MODA and a negative control (vehicle) was treated with ethanol solution.

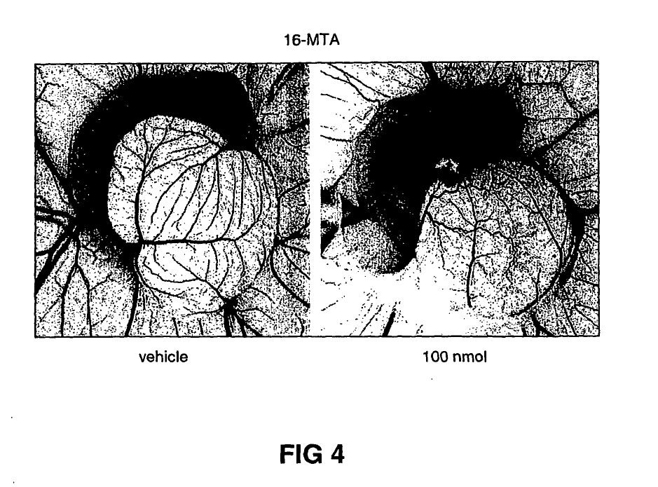

FIG. 4 shows that treatment with 100 nmol of 17-MODA yielded a reduction in the number of branching capillaries sprouting from the main vessels. In addition the vessel area is also diminished with the treatment. These results also demonstrate that 17-MODA was not cytotoxic to the embryo.

Quantitative measurement of the inhibitory effect of 17-MODA on angiogenesis in the CAM assay is shown in Table 8.

TABLE 8
Inhibitory effect of 17-MODA on angiogenesis in the CAM assay

| | Vehicle | 25 nmol | 50 nmol | 100 nmol |
|---|---|---|---|---|
| Vein length (%) | 100.0 ± 0.0 | 81.3 ± 16.8 | 112.8 ± 9.4 | 94.3 + 18.9 |
| Artery length (%) | 100.0 ± 0.0 | 82.6 ± 18.0 | 97.0 ± 11.7 | 97.2 ± 17.4 |
| Total vessel length (%) | 100.0 ± 0.0 | 81.9 ± 16.9 | 104.2 ± 10.4 | 95.9 ± 18.1 |
| Vein Diameter (%) | 100.0 ± 0.0 | 71.6 ± 13.4 | 81.3 ± 8.4 | 70.0 ± 15.0 |

(% of control; n = 6, Mean ± SEM)

As can be seen, even the lowest dose of 17-MODA inhibited vein length, artery length, total vessel length and vein diameter. The extent of inhibition increased with increasing dose of 17-MODA.

### EXAMPLE 12

Inhibition of Angiogenesis in a Mouse Corneal Vascularisation Model

(i) Materials and Methods

Unlike most mucosal surfaces, the normal cornea does not contain blood vessels. To induce neovascularisation in the cornea of mice, cornea were scratched and infected with Pseudomonas aeruginosa as essentially described in Cole, N., Willcox, M. D. P., Fleiszig, S. M. J., Stapleton, F., Bao, S., Tout, S., Husband, A. J. (1998) “Different strains of Pseudomonas aeruginosa isolated from ocular infections or inflammation display distinct corneal pathologies in an animal model.” Curr. Eye Res., 17:730-735.

Briefly, stock cultures of P. aeruginosa 6294 stored in 30% glycerol at −70° C. were inoculated into 10 mL of tryptone soya broth (Oxoid Ltd, Sydney, Australia). Cultures were prepared as previously described (Cole et al. (1998) Curr. Eye Res. 17:730-735) and suspended in phosphate buffered saline (PBS) to a concentration of 4×10⁸ cfu (colony forming units)/ml. Bacterial concentration was adjusted turbidimetrically and the dose confirmed retrospectively by viable counts.

Inbred 6-8 week old BALB/c mice were anaesthetised with Averin (125 mg/kg, intraperitoneally) and the corneal surfaces of the eyes were incised with a sterile 27 gauge needle. 5 μL of the bacterial suspension (2.0×10⁶ cfu) of strain 6294 was pipetted directly onto the wounded cornea of the left eye only. The right eye of each animal served as a control and was scratched but not infected. A minimum of eight mice per treatment group were used.

12-MTA at 200 μmol/10 μL was prepared as an emulsion in unpreserved paraffin and lanolin ophthalmic ointment base (Polyvisc, Alcon, Belgium) for topical application. Animals were divided into three treatment groups: Group 1 received no treatment; Group 2 received 10 μL of vehicle topically to the cornea per treatment to both the challenged and scratch control eye; and Group 3 received 10 μL of the 12-MTA as described above to both the challenged and scratch control eyes. The treatment schedule was begun four days after challenge and then every second day until the termination of the experiment 14 days post-challenge.

Mice were examined prior to bacterial challenge, immediately subsequent to bacterial challenge and 7 and 14 days post-challenge by a masked observer. The animals were anaesthetised for examination as described above and the corneas were examined at 48× magnification under white light using an FS2 photo slit-lamp biomicroscope (Topcon Corporation, Tokyo, Japan). At 7 and 14 days post-challenge, following the white light examination, 1% sodium fluorescein was instilled and the corneas viewed under UV light. Grades of severity of corneal damage were made and measurement of the extent and incursion of vessels into the central cornea were made.

Measurements were examined for significance using non-parametric Kruskal-Wallis and Mann Whitney U analysis.

For histological examination of corneas, mice were sacrificed at 14 days post-challenge. The eyes were immediately enucleated, fixed in neutral buffered formalin and embedded in paraffin. 5 μM sections were cut and stained with haematoxylin and eosin for histopathological examination.

(ii) Results

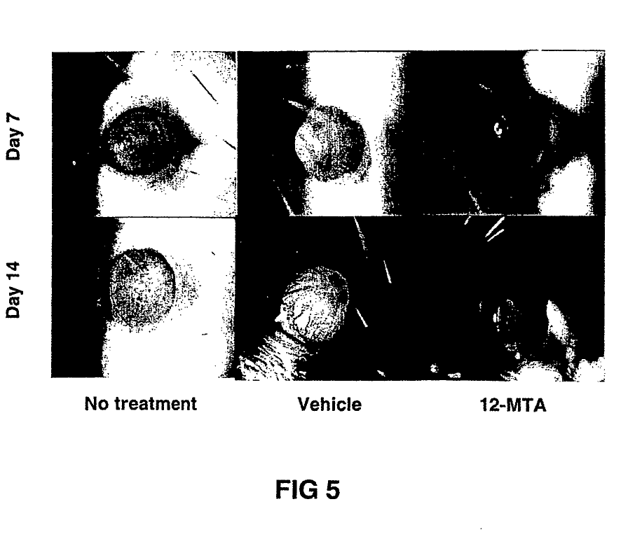

Photomicrographs of typical examples of mice in all three treatment-groups at Days 7 and 14 post-challenge are shown in FIG. 5. At Day 7, vascularisation to approximately 50% of the corneal diameter was observed in Groups 1 and 2. Group 3 showed reduced vascularisation as compared to Groups 1 and 2. Similarly, at Day 14, vascularisation to approximately 100% of the corneal diameter was observed in Groups 1 and 2. Group 3 showed reduced vascularisation as compared to Groups 1 and 2.

At 7 days post-challenge 7/7 mice (100%) in the group receiving no treatment (Group 1) and ⅝ (63%) of those receiving vehicle only (Group 2) showed vascularisation of the infected eye. However, only 4/10 (40%) of mice receiving 12-MTA treatment (Group 3) showed vascularisation. At 14 days post-challenge 6/7 mice (86%) in the group receiving no treatment (Group 1) and 6/8 (75%) of those receiving vehicle only (Group 2) showed vascularisation of the infected eye. Only 5/10 (50%) of mice receiving 12-MTA treatment (Group 3) showed vascularisation. This data is summarised in Table 9. The scratch control eyes in all groups showed no differences at any time point indicating that 12-MTA does not affect the cornea at this dose rate.

TABLE 9
Percentage of animals showing vascularisation in the infected eye
at 7 and 14 days post-challenge with P. aeruginosa 6294

| Vascularisation | Day 7 | Day 14 |
|---|---|---|
| No treatment | 7/7 (100%) | 6/7 (86%) |
| Vehicle | 5/8 (63%) | 6/8 (75%) |
| 12-MTA 200 μmole | 4/10 (40%) | 5/10 (50%) |

Grading the severity of corneal damage also showed that 12-MTA inhibited corneal neovascularization. In Group 2 the ocular responses were consistent within the group with a median score of 2.5 and a range of 1-4 with 25 % of animals showing a persistent epithelial defect. In Group 3 the ocular responses ranged from mild (50%) to severe (10%) with a median score 2.1 (range 0.54) and 20% of animals had a persistent epithelial defect. At Day 14 post-challenge the ocular responses in Group 1 ranged from mild (14%) to severe (70%) with a median score 3.3 (range 14). In Group 2 the ocular responses ranged from ranged from mild to moderate (63%) to severe (37%) with a median score of 3.3 (range 1-4). In Group 3 the ocular responses ranged from mild (60%) to severe (20%) with a median score 1.65 (range 0-4). No animals in any group had a persisting epithelial defect at this time.

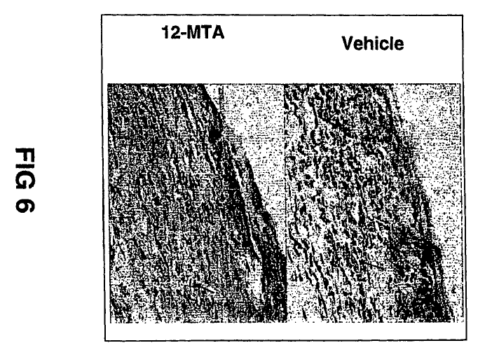

FIG. 6 shows histological examination of corneas treated with vehicle or 12-MTA for 14 days post-challenge (photomicrographs shown at 400× magnification). Arrows indicate blood vessels in the corneal stroma.

Histological examination of the corneas showed generalised blood vessel formation throughout the entire stroma in Groups 1 and 2. However, reduced blood vessel formation was observed for Group 3 at the same time.

Finally, it will be appreciated that various modifications and variations of the methods and compositions of the invention described herein will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the fields of vascular biology, pharmacology or related fields are intended to be within the scope of the present invention.

1. A method of inhibiting endothelial cell proliferation in a biological system, the method including the step of administering to the biological system an effective amount of an alkyl-substituted fatty acid, wherein the alkyl-substituted fatty acid is capable of inhibiting endothelial cell proliferation and the alkyl-substituted fatty acid has the following chemical formula: 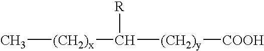
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

2. A method according to claim 1, wherein R is a methyl or ethyl group.

3. A method according to claim 1, wherein the alkyl-substituted fatty acid is 18-methylnonadecanoic acid, 17-methyloctadecanoic acid, 10-methyloctadecanoic acid, 16-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyidodecanoic acid, or any combination of these alkyl-substituted fatty acids.

4. A method according to claim 1, wherein the biological system is a human subject.

5. A method according to claim 4, wherein the proliferation of the endothelial cell is associated with uncontrolled or undesired angiogenesis.

6. A method according to claim 5, wherein the angiogenesis is associated with the formation or expansion of solid tumours, angiofibroma, corneal neovascularisation, retinal/choroidal neovascularization, arteriovenous malformations, arthritis, rheumatoid arthritis, lupus, connective tissue disorders, Osler-Weber syndrome, atherosclerotic plaques, psoriasis, pyogenic granuloma, retrolental fibroplasias, scleroderma, granulations, henagioma; trachoma, hemophilic joints, vascular adhesions, hypertrophic scars, diseases associated with chronic inflammation, sarcoidosis, inflammatory bowel diseases, Crohn's disease or ulcerative colitis.

7. A method according to claim 1, wherein the method further includes administering an effective amount of an immunosuppressant.

8. A method according to claim 7, wherein the immunosuppressant is cyclosporin A, rapamycin or FK506.

9. A method of inhibiting angiogenesis in a biological system, the method including the step of administering to the biological system an effective amount of an alkyl-substituted fatty acid, wherein the alkyl-substituted fatty acid is capable of inhibiting angiogenesis and the alkyl-substituted fatty acid has the following chemical formula: 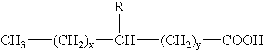
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

10. A method according to claim 9, wherein R is a methyl or ethyl group.

11. A method according to claim 9, wherein the alkyl-substituted fatty acid is 18-methylnonadecanoic acid, 17-methyloctadecanoic acid, 10-methyloctadecanoic acid, 16-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyldodecanoic acid, or any combination of these alkyl-substituted fatty acids.

12. A method according to claim 9, wherein the biological system is a human subject.

13. A method according to claim 12, wherein the angiogenesis is uncontrolled or undesired angiogenesis.

14. A method according to claim 13, wherein the angiogenesis is associated with the formation or expansion of solid tumours, angiofibroma, corneal neovascularisation, retinal/choroidal neovascularization, arteriovenous malformations, arthritis, rheumatoid arthritis, lupus, connective tissue disorders, Osler-Weber syndrome, atherosclerotic plaques, psoriasis, pyogenic granuloma, retrolental fibroplasias, scleroderma, granulations, henagioma; trachoma, hemophilic joints, vascular adhesions, hypertrophic scars, diseases associated with chronic inflammation, sarcoidosis, inflammatory bowel diseases, Crohn's disease or ulcerative colitis.

15. A method according to claim 9, wherein the method further includes administering an effective amount of an immunosuppressant.

16. A method according to claim 15, wherein the immunosuppressant is cyclosporin A, rapamycin or FK506.

17. A method of inhibiting neovascularisation of a cornea, the method including the step of administering to the cornea an effective amount of an alkyl-substituted fatty acid, wherein the alkyl-substituted fatty acid is capable of inhibiting neovascularisation in the cornea and the alkyl-substituted fatty acid has the following chemical formula: 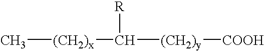
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

18. A method according to claim 17, wherein R is a methyl or ethyl group.

19. A method of reducing the amount of an anti-angiogenic agent administered to a biological system to achieve a desired level of inhibition of angiogenesis, the method including the step of administering to the biological system an effective amount of an alkyl-substituted fatty acid, wherein the alkyl-substituted fatty acid has the following chemical formula: 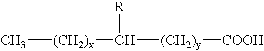
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

20. A method according to claim 19, wherein R is a methyl or ethyl group.

21. A method according to claim 19, wherein the alkyl-substituted fatty acid is 18-methylnonadecanoic acid, 17-methyloctadecanoic acid, 10-methyloctadecanoic acid, 16-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyidodecanoic acid, or any combination of these alkyl-substituted fatty acids.

22. A method according to claim 19, wherein the biological system is a human subject.

23. A method according to claim 22, wherein the angiogenesis is uncontrolled or undesired angiogenesis.

24. A method according to claim 23, wherein the angiogenesis is associated with the formation or expansion of solid tumours, angiofibroma, corneal neovascularisation, retinal/choroidal neovascularization, arteriovenous malformations, arthritis, rheumatoid arthritis, lupus, connective tissue disorders, Osler-Weber syndrome, atherosclerotic plaques, psoriasis, pyogenic granuloma, retrolental fibroplasias, scleroderma, granulations, henagioma; trachoma, hemophilic joints, vascular adhesions, hypertrophic scars, diseases associated with chronic inflammation, sarcoidosis, inflammatory bowel diseases, Crohn's disease or ulcerative colitis.

25. A pharmaceutical composition that inhibits endothelial cell proliferation and/or angiogenesis, the composition including an alkyl-substituted fatty acid with the following chemical formula: 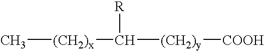
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

26. A pharmaceutical composition according to claim 25, wherein R is a methyl or ethyl group.

27. A pharmaceutical composition according to claim 25, wherein the alkyl-substituted fatty acid is 18-methylnonadecanoic acid, 17-methyloctadecanoic acid, 10-methyloctadecanoic acid, 16-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyldodecanoic acid, or any combination of these alkyl-substituted fatty acids.

28. A pharmaceutical composition according to claim 25, wherein the composition further includes an immunosuppressant.

29. A pharmaceutical composition according to claim 28, wherein the immunosuppressant is cyclosporin A, rapamycin or FK506.

30. A pharmaceutical composition including an alkyl-substituted fatty acid and an immunosuppressant, wherein the alkyl-substituted fatty acid has the following chemical formula: 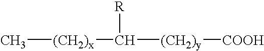
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

31. A pharmaceutical composition according to claim 30, wherein R is a methyl or ethyl group.

32. A pharmaceutical composition according to claim 30, wherein the alkyl-substituted fatty acid is 18-methylnonadecanoic acid, 17-methyloctadecanoic acid, 10-methyloctadecanoic acid, 16-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyldodecanoic acid, or any combination of these alkyl-substituted fatty acids.

33. A pharmaceutical composition according to claim 30, wherein the immunosuppressant is cyclosporin A, rapamycin or FK506.

34. A use of an alkyl-substituted fatty acid for the preparation of a medicament that inhibits endothelial cell proliferation and/or inhibits angiogenesis, wherein the alkyl-substituted fatty acid has the following chemical formula: 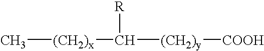
or a salt thereof, wherein:
R is an alkyl group of 1 to 6 carbon atoms;
x is equal to or greater than 0, y is equal to or greater than 0, and x+y is between 0 and 46 for saturated alkyl-substituted fatty acids; and
for unsaturated alkyl-substituted fatty acids x or y is equal to or greater than 2, at least one CH₂—CH₂ group in (CH₂)ₓ and/or (CH₂)_{y} is replaced with a CH═CH group or a C≡C group, and x+y is between 2 and 46.

35. A use according to claim 34, wherein R is a methyl or ethyl group.

36. A use according to claim 34, wherein the alkyl-substituted fatty acid is 18-methylnonadecanoic acid, 17-methyloctadecanoic acid, 10-methyloctadecanoic acid, 16-methylheptadecanoic acid, 15-methylheptadecanoic acid, 15-methylhexadecanoic acid, 14-methylhexadecanoic acid, 14-methylpentadecanoic acid, 13-methylpentadecanoic acid, 13-methyltetradecanoic acid, 12-methyltetradecanoic acid, 12-methyltridecanoic acid, 11-methyltridecanoic acid, 11-methyldodecanoic acid, 10-methyldodecanoic acid, or any combination of these alkyl-substituted fatty acids.

37. A use according to claim 34, wherein the medicament further includes an immunosuppressant.

38. A use according to claim 37, wherein the immunosuppressant is cyclosporin A, rapamycin or FK506.
